(19) United States
(12) Patent Application Publication
(10) Pub. No.: US 2016/0090485 A1
(43) Pub. Date: Mar. 31, 2016

(54) Method for Preparing Urethane (Meth)Acrylates

(71) Applicant: BASF SE, Ludwigshafen (DE)

(72) Inventors: Reinhold Schwalm, Wachenheim (DE); Susanne Neumann, Speyer (DE); Delphine Kimpel, Speyer (DE); Erich Beck, Ladenburg (DE); Klaus Menzel, Ludwigshafen (DE)

(21) Appl. No.: 14/892,371

(22) PCT Filed: May 16, 2014
(86) PCT No.: PCT/EP2014/060079

§ 371 Date: Nov. 19, 2015

(30) Foreign Application Priority Data
May 27, 2013 (EP) 13169361.6

(51) Int. Cl. C09D 4/00; C07C 269/02; C07C 271/20

(52) U.S. Cl. CPC C09D 4/00; C07C 271/20; C07C 269/02

(57) ABSTRACT

Described is a process for preparing urethane (meth)acrylates. In a first step, a hydroxyalkyl (meth)acrylate (A) is reacted with a lactone (B) in the presence of at least one catalyst (C), selected from the group consisting of iron compounds, titanium compounds, aluminum compounds, zirconium compounds, manganese compounds, nickel compounds, zinc compounds, cobalt compounds, and bismuth compounds to provide a product; and, in a further step, the product is reacted with a polyisocyanate (D) which comprises at least one hydroxyalkyl (meth)acrylate bonded via an allophanate group.

### CROSS-REFERENCE TO RELATED APPLICATIONS

This is application is the National Stage Entry of PCT/EP2014/060079 filed May 16, 2014, which claims priority to European Patent Application No. 13169361.6, filed May 27, 2013, the disclosures of which are incorporated herein by reference in their entireties.

### TECHNICAL FIELD

The present invention describes a new process for preparing urethane (meth)acrylates.

### BACKGROUND

Urethane acrylates based on caprolactone-modified resins are known from U.S. Pat. No. 4,188,472, for example. In DE 2939584 (=U.S. Pat. No. 4,188,472), 2-hydroxyethyl acrylate and epsilon-caprolactone are reacted together ring-openingly in the presence of various catalysts based on titanium or tin or on organic acids (sulfuric acid, p-toluenesulfonic acid), and the product obtained is subsequently reacted with diisocyanates to give the urethane.

DE 10246512 describes preparing low-viscosity polyisocyanates by reacting polyisocyanates containing oxadiazinetrione groups with alcohols which comprise at least one double bond that is polymerizable by electromagnetic radiation.

WO 07/059011 and WO 07/059070 describe urethane (meth)acrylates with allophanate groups that comprise fluorinated alcohols in incorporated form. The (meth)acrylate groups are incorporated in each case via urethane groups.

EP 783008 describes urethane (meth)acrylates obtained through reaction of polyisocyanates with alcohols containing (meth)acrylate groups. The (meth)acrylate groups are incorporated in each case via urethane groups.

### SUMMARY

A first aspect of the present invention is directed to a urethane (meth)acrylate. In a first embodiment, a urethane (meth)acrylate is of the formula (I)

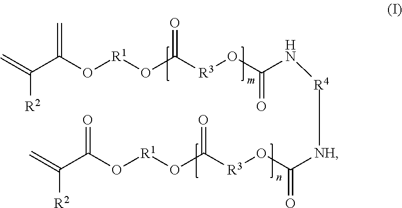

wherein R¹ is a divalent alkylene radical having 2 to 12 carbon atoms and, optionally substituted with C₁ to C₄ alkyl groups and/or interrupted by one or more oxygen atoms, said radical having preferably 2 to 10 carbon atoms, more preferably 2 to 8, and very preferably 3 to 6 carbon atoms, R² in each case independently of any other is methyl or hydrogen, preferably hydrogen, R³ is a divalent alkylene radical having 1 to 12 carbon atoms and optionally substituted with C₁ to C₄ alkyl groups and/or interrupted by one or more oxygen atoms, said radical having preferably 2 to 10, more preferably 3 to 8, and very preferably 3 to 4 carbon atoms, R⁴ is a divalent organic radical formed by conceptual abstraction of two isocyanate groups from a polyisocyanate (D) which comprises at least one hydroxyalkyl (meth)acrylate bonded via an allophanate group, and n and m independently of one another are positive numbers from 1 to 5, preferably 2 to 5, more preferably 2 to 4, very preferably 2 to 3, and more particularly 2 to 2.5.

In a second embodiment, the urethane (meth)acrylate of the first embodiment is modified, wherein R¹ is selected from the group consisting of 1,2-ethylene, 1,2- or 1,3-propylene, 1,2-, 1,3-, or 1,4-butylene, 1,1-dimethyl-1,2-ethylene, 1,2-dimethyl-1,2-ethylene, 1,5-pentylene, 1,6-hexylene, 1,8-octylene, 1,10-decylene, and 1,12-dodecylene.

In a third embodiment, the urethane (meth)acrylate of the first and second embodiments is modified, wherein R³ is selected from the group consisting of methylene, 1,2-ethylene, 1,2-propylene, 1,3-propylene, 1,2-butylene, 1,3-butylene, 1,4-butylene, 1,5-pentylene, 1,5-hexylene, 1,6-hexylene, 1,8-octylene, 1,10-decylene, 1,12-dodecylene, 2-oxa-1,4-butylene, 3-oxa-1,5-pentylene, or 3-oxa-1,5-hexylene.

In a fourth embodiment, the urethane (meth)acrylate the first through third embodiments is modified, wherein the catalyst is a titanium compound, zinc compound, or bismuth compound.

A second aspect of the present invention is directed to a process. In a fifth embodiment, a process for preparing the urethane (meth)acrylate of the first through fourth embodiments comprises: in a first step reacting a hydroxyalkyl (meth)acrylate (A) of the formula

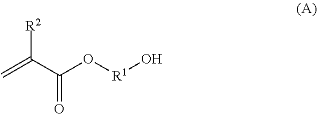

with a lactone (B) of the formula

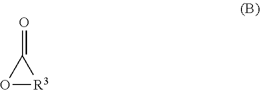

in the presence of at least one catalyst (C), selected from the group consisting of iron compounds, titanium compounds, aluminum compounds, zirconium compounds, manganese compounds, nickel compounds, zinc compounds, cobalt compounds, and bismuth compounds to provide a product; and, in a further step, reacting the product from the first step with a polyisocyanate (D) which comprises at least one hydroxyalkyl (meth)acrylate bonded via an allophanate group.

In a sixth embodiment, the process of the fifth embodiment is modified, wherein the polyisocyanate (D) is obtained by reacting at least one (cyclo)aliphatic diisocyanate with at least one hydroxyalkyl (meth)acrylate in the presence of at least one catalyst able to accelerate the formation of allophanate groups.

In a seventh embodiment, the process of the sixth embodiment is modified, wherein the diisocyanate is selected from the group consisting of hexamethylene 1,6-diisocyanate, isophorone diisocyanate, and 4,4′- or 2,4′-di(isocyanatocyclohexyl)methane.

In an eighth embodiment, the process of the sixth and seventh embodiment is modified, wherein the at least one hydroxyalkyl (meth)acrylate used to prepared component (D) is selected from the group consisting of 2-hydroxyethyl (meth)acrylate, 2- or 3-hydroxypropyl (meth)acrylate, 1,4-butanediol mono(meth)acrylate, neopentyl glycol mono(meth)acrylate, 1,5-pentanediol mono(meth)acrylate, and 1,6-hexanediol mono(meth)acrylate.

In a ninth embodiment, the process of the fifth embodiment is modified, wherein the polyisocyanate (D) comprises a compound of the formula

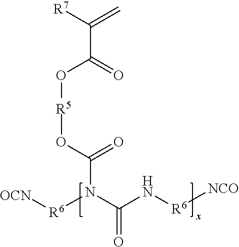

wherein R⁵ is a divalent alkylene radical having 2 to 12 carbon atoms and optionally substituted with C₁ to C₄ alkyl groups and/or interrupted by one or more oxygen atoms, said radical having preferably 2 to 10 carbon atoms, more preferably 2 to 8, and very preferably 3 to 6 carbon atoms, R⁶ is a divalent alkylene or cycloalkylene radical having 2 to 20 carbon atoms and optionally substituted with C₁ to C₄ alkyl groups and/or interrupted by one or more oxygen atoms, said radical having preferably 4 to 15 carbon atoms, more preferably 6 to 13 carbon atoms, R⁷ is hydrogen or methyl, preferably hydrogen, and X is a positive number which on average is 2 up to 6, preferably from 2 to 4.

In a tenth embodiment, the process of the ninth embodiment is modified, wherein R⁵ is selected from the group consisting of 1,2-ethylene, 1,2- or 1,3-propylene, 1,2-, 1,3- or 1,4-butylene, 1,1-dimethyl-1,2-ethylene, 1,2-dimethyl-1,2-ethylene, 1,5-pentylene, 1,6-hexylene, 1,8-octylene, 1,10-decylene, and 1,12-dodecylene.

In an eleventh embodiment, the process of the ninth and tenth embodiments is modified, wherein R⁶ is selected from the group consisting of 1,6-hexylene,

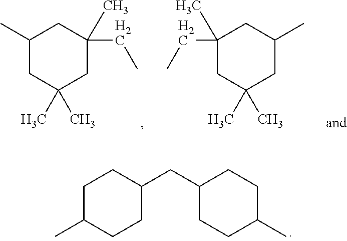

A third aspect of the invention is directed to a coating material In a twelfth embodiment, t radiation-curable coating material comprises at least one urethane (meth)acrylate of the first through fourth embodiments and, optionally, at least one radically polymerizable compound and, also optionally, at least one photoinitiator.

A fourth aspect of the present invention is directed to a use. In a thirteenth embodiment, the radiation-curable coating material of the twelfth embodiment is used to coat wood, paper, textile, leather, nonwoven, plastics surfaces, PVC, glass, ceramic, mineral building materials, molded cement blocks, fiber cement slabs, metals, or coated-metal substrates.

A fifth aspect of the present invention is directed to a method. In a fourteenth embodiment, a method of coating a substrate comprises applying the radiation-curable coating material of the twelfth embodiment to a substrate selected from the group consisting of wood, paper, textile, leather, nonwoven, plastics surfaces, PVC, glass, ceramic, mineral building materials, molded cement blocks, fiber cement slabs, metals, or coated-metal substrates.

### DETAILED DESCRIPTION

Described are urethane (meth)acrylates which unite good scratch resistance, good elasticity, and low viscosity with one another.

Specifically, the present invention provides urethane (meth)acrylates of the formula (I)

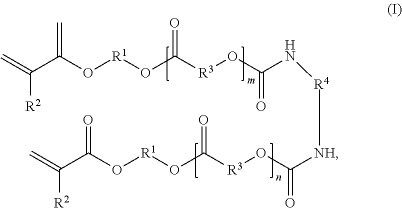

wherein
R¹ is a divalent alkylene radical which has 2 to 12 carbon atoms and may optionally be substituted by C₁ to C₄ alkyl groups and/or interrupted by one or more oxygen atoms, said radical having specifically 2 to 10 carbon atoms, more specifically 2 to 8, and very specifically 3 to 6 carbon atoms,
R² in each case independently of any other is methyl or hydrogen, specifically hydrogen,
R³ is a divalent alkylene radical which has 1 to 12 carbon atoms and may optionally be substituted by C₁ to C₄ alkyl groups and/or interrupted by one or more oxygen atoms, said radical having specifically 2 to 10, more specifically 3 to 8, and very specifically 3 to 4 carbon atoms,
R⁴ is a divalent organic radical which is formed by conceptual abstraction of two isocyanate groups from a polyisocyanate (D) which comprises at least one hydroxyalkyl (meth)acrylate bonded via an allophanate group, and
   n and m independently of one another are positive numbers from 1 to 5, specifically 2 to 5, more specifically 2 to 4, very specifically 2 to 3, and more particularly 2 to 2.5.

In one or more embodiments, the double bond density of the urethane (meth)acrylate of the invention, measured in mol of (meth)acrylate groups per kg of urethane (meth)acrylate, is generally 2 to 4 mol/kg, specifically 2.4 to 3.4, and more specifically 2.6 to 3.0 mol/kg.

The present invention further provides a process for preparing such urethane (meth)acrylates by in a first step reacting together a hydroxyalkyl (meth)acrylate (A) of the formula

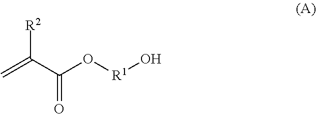

with a lactone (B) of the formula

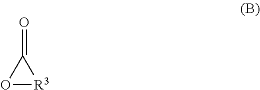

in the presence of at least one catalyst (C), selected from the group consisting of iron compounds, titanium compounds, aluminum compounds, zirconium compounds, manganese compounds, nickel compounds, zinc compounds, cobalt compounds, and bismuth compounds,
and in a further step reacting the resulting product from the first step with a polyisocyanate (D) which comprises at least one hydroxyalkyl (meth)acrylate bonded via an allophanate group.

The values for n and m may on average also adopt uneven values, but in that case are of course even relative to each individual molecule of the formula above.

For the purposes of this specification, C₁-C₄ alkyl is methyl, ethyl, n-propyl, isopropyl, n-butyl, isobutyl, sec-butyl, or tert-butyl, specifically methyl, ethyl, and n-butyl, and more specifically methyl.

Examples of the radical R¹ are 1,2-ethylene, 1,2- or 1,3-propylene, 1,2-, 1,3- or 1,4-butylene, 1,1-dimethyl-1,2-ethylene, 1,2-dimethyl-1,2-ethylene, 1,5-pentylene, 1,6-hexylene, 1,8-octylene, 1,10-decylen, or 1,12-dodecylene. Preference is given to 1,2-ethylene, 1,2- or 1,3-propylene, 1,4-butylene, and 1,6-hexylene, particular preference to 1,2-ethylene, 1,2-propylene, and 1,4-butylene, and special preference to 1,2-ethylene.

Examples of the radical R³ are methylene, 1,2-ethylene, 1,2-propylene, 1,3-propylene, 1,2-butylene, 1,3-butylene, 1,4-butylene, 1,5-pentylene, 1,5-hexylene, 1,6-hexylene, 1,8-octylene, 1,10-decylene, 1,12-dodecylene, 2-oxa-1,4-butylene, 3-oxa-1,5-pentylene, or 3-oxa-1,5-hexylene, specifically 1,3-propylene, 1,4-butylene, 1,5-pentylene, 1,5-hexylene, and 1,12-dodecylene, more specifically 1,5-pentylene.

In accordance with the invention in the first step hydroxyalkyl (meth)acrylates (A) of the formula

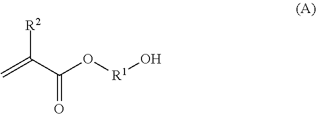

in which R¹ and R² have the definitions set out above are reacted with (n+m)/2 equivalents of lactone (B) of the formula

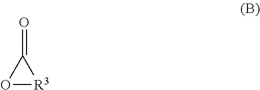

in which R³ has the definitions set out above, to give an intermediate of the formula

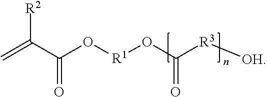

In one or more embodiments, the hydroxyalkyl (meth)acrylates (A) are 2-hydroxyethyl (meth)acrylate, 2- or 3-hydroxypropyl (meth)acrylate, 1,4-butanediol mono(meth)acrylate, neopentyl glycol mono(meth)acrylate, 1,5-pentanediol mono(meth)acrylate, and 1,6-hexanediol mono(meth)acrylate, very specifically 2-hydroxyethyl (meth)acrylate, 2-hydroxypropyl (meth)acrylate, and 1,4-butanediol mono(meth)acrylate, more particularly 2-hydroxyethyl (meth)acrylate.

In one or more embodiments, acrylates are used instead of the methacrylates here.

In one or more embodiments, the formula of the lactone (B) is as follows:

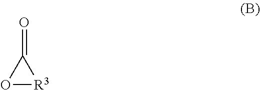

In one or more embodiments, the lactones are beta-propiolactone, gamma-butyrolactone, gamma-ethyl-gamma-butyrolactone, gamma-valerolactone, delta-valerolactone, epsilon-caprolacton, 7-methyloxepan-2-one, 1,4-dioxepan-5-one, oxacyclotridecan-2-one, and 13-butyl-oxacyclotridecan-2-one.

In one or more specific embodiments, the lactones are gamma-butyrolactone, delta-valerolactone, and epsilon-caprolactone; particularly epsilon-caprolactone.

The reaction in the first step takes place in the presence of at least one catalyst (C), with tin compounds being excluded in accordance with the invention.

In one or more embodiments, the catalysts (C) are selected from the group consisting of compounds of iron, titanium, aluminum, zirconium, manganese, nickel, zinc, cobalt, and bismuth, specifically compounds of titanium, aluminum, zirconium, zinc, or bismuth, more specifically compounds of titanium, zinc, or bismuth, very specifically compounds of titanium or bismuth, and more particularly bismuth compounds.

Possible for example are metal complexes such as acetylacetonates of iron, of titanium, of aluminum, of zirconium, of manganese, of nickel, of zinc, and of cobalt.

Examples of compounds used as zirconium, bismuth, titanium, and aluminum compounds include the following: zirconium tetraacetylacetonate (e.g., K-KAT® 4205 from King Industries); zirconium dionates (e.g., K-KAT® XC-9213; XC-A 209 and XC-6212 from King Industries); and aluminum dionate (e.g., K-KAT® 5218 from King Industries).

Zinc compounds contemplated in this context are those in which the following anions are used: F⁻, Cl⁻, ClO⁻, ClO₃⁻, ClO₄⁻, Br⁻, I⁻, IO₃⁻, CN⁻, OCN⁻, NO₂⁻, NO₃⁻, HCO₃⁻, CO₃²⁻, S²⁻, SH⁻, HSO₃⁻, SO₃²⁻, HSO₄⁻, SO₄²⁻, S₂O₂²⁻, S₂O₄²⁻, S₂O₅²⁻, S₂O₆²⁻, S₂O₇²⁻, S₂O₅²⁻, H₂PO₂⁻, H₂PO₄⁻, HPO₄²⁻, PO₄³⁻, P₂O₇⁴⁻, (OCₙH₂ₙ₊₁)⁻, (CₙH₂ₙ₋₁O₂)⁻, (CₙH₂ₙ₋₃O₂)⁻, and (Cₙ₊₁H₂ₙ₋₂O₄)²⁻, where n stands for the numbers 1 to 20. In one or more embodiments, carboxylates where the anion conforms to the formulae (CₙH₂ₙ₋₁O₂)⁻ and also (Cₙ₊₁H₂ₙ₋₂O₄)²⁻ with n being 1 to 20 are used. In one or more specific embodiments, the salts have monocarboxylate anions of the general formula (CₙH₂ₙ₋₁O₂)⁻ where n stands for the numbers 1 to 20. Especially noteworthy in this context are formate, acetate, propionate, hexanoate, neodecanoate, and 2-ethylhexanoate.

In one or more embodiments, the zinc catalysts are zinc carboxylates, more specifically those of carboxylates having at least six carbon atoms, very specifically at least eight carbon atoms, more particularly zinc(II) diacetate, zinc(II) dioctoate, or zinc(II) neodecanoate. Examples of commercial catalysts include Borchi® Kat 22 from OMG Borchers GmbH, Langenfeld, Germany.

In one or more embodiments, the titanium compounds are titanium tetraalcoholates Ti(OR)₄, more specifically those of alcohols ROH having 1 to 8 carbon atoms, examples being methanol, ethanol, isopropanol, n-propanol, n-butanol, isobutanol, sec-butanol, tert-butanol, n-hexanol, n-heptanol, and n-octanol, specifically methanol, ethanol, isopropanol, n-propanol, n-butanol, or tert-butanol, more specifically isopropanol and n-butanol.

As catalyst (C), preference is given to using at least one bismuth compound, as for example one to three, specifically one or two, and more specifically precisely one compound of bismuth in the +3 oxidation state.

Bismuth compounds (C) contemplated in this context are compounds of bismuth with the following anions: F⁻, Cl⁻, ClO⁻, ClO₃⁻, ClO₄⁻, Br⁻, I⁻, IO₃⁻, CN⁻, OCN⁻, NO₂⁻, NO₃⁻, HCO₃⁻, CO₃²⁻, S²⁻, SH⁻, HSO₃⁻, SO₃²⁻, HSO₄⁻, SO₄²⁻, S₂O₂²⁻, S₂O₄²⁻, S₂O₈²⁻, S₂O₈²⁻, S₂O₇²⁻, S₂O₈²⁻, H₂PO₂⁻, H₂PO₄⁻, HPO₄²⁻, PO₄³⁻, P₂O₇⁴⁻, (OCₓH₂ₓ₊₁)⁻, (CₓH₂ₓ₋₁O₂)⁻, (CₓH₂ₓ₋₃O₂)⁻, and (Cₓ₊₁H₂ₓ₋₂O₄)²⁻, where x stands for the numbers 1 to 20. In one or more embodiments, carboxylates where the anion conforms to the formulae (CₓH₂ₓ₋₁O₂)⁻ and also (Cₓ₊₁H₂ₓ₋₂O₄)²⁻ with x being 1 to 20 are used. In one more specific embodiments, the salts have monocarboxylate anions of the general formula (CₓH₂ₓ₋₁O₂)⁻ where x stands for the numbers 1 to 20, specifically 1 to 10. Especially noteworthy in this context are formate, acetate, propionate, hexanoate, neodecanoate, and 2-ethylhexanoate.

In one or more embodiments, the bismuth catalysts are bismuth carboxylates, more specifically those of carboxylates which have at least six carbon atoms, more particularly bismuth octoates, ethylhexanoates, neodecanoates, or pivalates; examples are K-KAT 348, XC-B221; XC-C227, XC 8203, and XK-601 from King Industries, TIB KAT 716, 716LA, 716XLA, 718, 720, and 789 from TIB Chemicals, and those from Shepherd Lausanne, and also, for example, Borchi® Kat 24, 315, and 320 from OMG Borchers GmbH, Langenfeld, Germany.

Mixtures of different metals may also be relevant in this context, such as, for example, in Borchi® Kat 0245 from OMG Borchers GmbH, Langenfeld, Germany.

In one or more specific embodiments, however, bismuth neodecanoate, bismuth 2-ethylhexanoate, and zinc 2-ethylhexanoate are used.

It is possible to boost the effect of the catalysts additionally through the presence of acids, as for example through acids having a pKa of <2,5, as described in EP 2316867 A1, or with a pKa of between 2.8 and 4.5, as described in WO 04/029121 A1. In one or more embodiments, acids with a pKa of not more than 4.8, more specifically of not more than 2.5 are used.

The polyisocyanate (D) is polyisocyanate (D) comprising at least one hydroxyalkyl (meth)acrylate bonded via an allophanate group.

Examples of such polyisocyanates are described for example in WO 00/39183 A1, particularly from page 4 line 17 to page 6 line 6 and products 1 to 12 in table 1 therein. The polyisocyanates (D) are preparable in the manner described therein from page 8 line 44 to page 10 line 2. These disclosures are each considered by reference to be part of the present description.

In one or more embodiments, the polyisocyanates (D) are those obtainable by reacting at least one (cyclo)aliphatic diisocyanate with at least one hydroxyalkyl (meth)acrylate in the presence of at least one catalyst able to accelerate the formation of allophanate groups.

(Cyclo)aliphatic in this specification stands for aliphatic or cycloaliphatic, specifically aliphatic.

Examples of (cyclo)aliphatic diisocyanates are aliphatic diisocyanates such as tetramethylene diisocyanate, hexamethylene diisocyanate (1,6-diisocyanatohexane), octamethylene diisocyanate, decamethylene diisocyanate, dodecamethylene diisocyanate, tetradecamethylene diisocyanate, lysine diisocyanate derivatives, tetramethylxylylene diisocyanate, trimethylhexane diisocyanate, or tetramethylhexane diisocyanate, cycloaliphatic diisocyanates such as 1,4-, 1,3- or 1,2-diisocyanatocyclohexane, 4,4′- or 2,4′-di(isocyanatocyclohexyl)methane, 1-isocyanato-3,3,5-trimethyl-5-(isocyanatomethyl)cyclohexane (isophorone diisocyanate), 1,3- or 1,4-bis(isocyanatomethyl)cyclohexane, or 2,4- or 2,6-diisocyanato-1-methylcyclohexane.

In one or more embodiments, the polyisocyanates (D) are hexamethylene 1,6-diisocyanate, isophorone diisocyanate, and 4,4′- or 2,4′-di(iso-cyanatocyclohexyl)methane, specifically hexamethylene 1,6-diisocyanate, isophorone diisocyanate, and 4,4′-di(isocyanatocyclohexyl)methane; very specifically hexamethylene 1,6-diisocyanate and isophorone diisocyanate, and more particularly hexamethylene 1,6-diisocyanate.

Hydroxyalkyl (meth)acrylates may be those as described above in relation to component (A), but may be different from the component (A) used. In one specific embodiment of the present invention, the hydroxyalkyl (meth)acrylate used as component (A) and the hydroxyalkyl (meth)acrylate used for component (D) are the same.

The hydroxyalkyl (meth)acrylates used for component (D) are specifically 2-hydroxyethyl (meth)acrylate, 2- or 3-hydroxypropyl (meth)acrylate, 1,4-butanediol mono(meth)acrylate, neopentyl glycol mono(meth)acrylate, 1,5-pentanediol mono(meth)acrylate, and 1,6-hexanediol mono(meth)acrylate, very specifically 2-hydroxyethyl (meth)acrylate, 2-hydroxypropyl (meth)acrylate, and 1,4-butanediol mono(meth)acrylate, more particularly 2-hydroxyethyl (meth)acrylate.

Catalysts able to accelerate the formation of allophanate groups are, for example, organozinc compounds, such as zinc acetylacetonate or zinc 2-ethylcaproate, or tetraalkylammonium compounds, such as, specifically, tetraalkylammonium hydroxides, carboxylates, and carbonates; particularly N,N,N-trimethyl-N-benzylammonium hydroxide, N,N,N-trimethyl-N-2-hydroxypropylammonium hydroxide, N,N,N-trimethyl-N-2-hydroxypropylammonium 2-ethylhexanoate, and N,N,N-trimethyl-N-2-hydroxypropylammonium formate, very specifically N,N,N-trimethyl-N-2-hydroxypropylammonium 2-ethylhexanoate.

Component (D) comprises allophanate groups in an allophanate group (calculated as C₂N₂HO₃=101 g/mol) content of 1 to 28 wt %, specifically of 3 to 25 wt %.

In one specific embodiment of the present invention at least 20 mol % of the hydroxyalkyl (meth)acrylates, specifically at least 25 mol %, more specifically at least 30 mol %, very specifically at least 35 mol %, more particularly at least 40 mol %, and especially at least 50 mol %, are bonded via allophanate groups.

In one specific embodiment, the polyisocyanate (D) which comprises at least one hydroxyalkyl (meth)acrylate bonded via an allophanate group comprises compounds of the formula

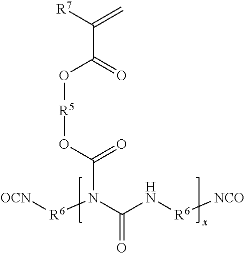

in which
R⁵ is a divalent alkylene radical which has 2 to 12 carbon atoms and may optionally be substituted by C₁ to C₄ alkyl groups and/or interrupted by one or more oxygen atoms, said radical having specifically 2 to 10 carbon atoms, more specifically 2 to 8, and very specifically 3 to 6 carbon atoms,
R⁶ is a divalent alkylene or cycloalkylene radical which has 2 to 20 carbon atoms and may optionally be substituted by C₁ to C₄ alkyl groups and/or interrupted by one or more oxygen atoms, said radical having specifically 4 to 15 carbon atoms, more specifically 6 to 13 carbon atoms,
R⁷ is hydrogen or methyl, specifically hydrogen, and
x is a positive number which on average is 2 up to 6, specifically from 2 to 4.

In one or more embodiments, the polyisocyanate (D) represented in this formula constitutes, by conceptual abstraction of two isocyanate groups, a radical R⁴ according to the formula for the urethane (meth)acrylate of the invention.

Examples of the radical R⁵ are 1,2-ethylene, 1,2- or 1,3-propylene, 1,2-, 1,3- or 1,4-butylene, 1,1-dimethyl-1,2-ethylene, 1,2-dimethyl-1,2-ethylene, 1,5-pentylene, 1,6-hexylene, 1,8-octylene, 1,10-decylen, or 1,12-dodecylene. Preference is given to 1,2-ethylene, 1,2- or 1,3-propylene, 1,4-butylene, and 1,6-hexylene, particular preference to 1,2-ethylene, 1,2-propylene, and 1,4-butylene, and special preference to 1,2-ethylene.

Specifically, R⁶ is selected from the group consisting of 1,6-hexylene,

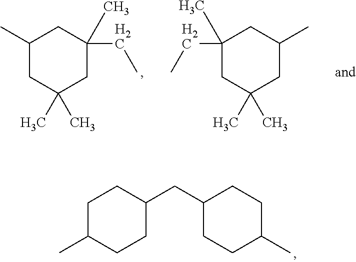

and more specifically it is 1,6-hexylene.

In one specific embodiment of the present invention, R⁶ is 1,6-hexylene and R⁵ is selected from the group consisting of 1,2-ethylene, 1,2-propylene, and 1,4-butylene, specifically of 1,2-ethylene and 1,4-butylene, and more specifically 1,2-ethylene.

A commercially available polyisocyanate with R⁵=1,2-ethylene, R⁶=1,6-hexylene, and R⁷=hydrogen is available under the trade name Laromer® LR9000 from BASF SE, Ludwigshafen with an NCO content of 14.5-15.5 wt %.

The process of the invention for preparing the urethane (meth)acrylates may be implemented as follows:

The reaction of components (A) and (B) takes place specifically at temperatures of 50 to 150° C., specifically 70 to 130° C., over a period of 3 to 20 hours, specifically of 5 to 12 hours, with stirring or pumped circulation.

In this reaction, components (A) and (B) are mixed with one another in the desired stoichiometry (mol:mol), which is specifically 1:1.5 to 3, more specifically 1:1.8 to 2.5, very specifically 1:2 to 2.3, and more particularly 1:2, and the mixture is heated. It is also possible for component (A) to be introduced initially and for component (B) to be added not until during or after the heating procedure.

Before, during, or after the heating procedure, the catalyst (C), optionally divided into a number of portions, is added to the mixture.

It is also possible first to react component (A) with only part of the compound (B), and to add the remainder of the compound (B) to the reaction at a later point in time.

In one or more embodiments, all three components, (A), (B), and (C), are mixed with one another and jointly heated and reacted.

The catalyst (C) is added to the reaction mixture generally in amounts of 0.001 to 2 wt %, based on the sum of components (A) and (B), specifically 0.005 to 1.5 wt %, more specifically 0.01 to 1, and very specifically 0.01 to 0.5 wt %.

It is optionally possible, although less desired, for the reaction to be carried out in the presence of at least one solvent.

Examples of such solvents are aromatic (including alkylated benzenes and naphthalenes) and/or (cyclo)aliphatic hydrocarbons and mixtures thereof, chlorinated hydrocarbons, ketones, esters, alkoxylated alkanoic acid alkyl esters, ethers, or mixtures of the solvents.

In one or more embodiments, the aromatic hydrocarbon mixtures are those which comprise primarily aromatic C₇ to C₁₄ hydrocarbons and which may span a boiling range from 110 to 300° C., particular preference being given to toluene, o-, m-, or p-xylene, trimethylbenzene isomers, tetramethylbenzene isomers, ethylbenzene, cumene, tetrahydronaphthalene, and mixtures comprising them.

Examples thereof are the Solvesso® products from ExxonMobil Chemical, particularly Solvesso® 100 (CAS No. 64742-95-6, primarily C9 and C10 aromatics, boiling range about 154-178° C.), 150 (boiling range about 182-207° C.), and 200 (CAS No. 64742-94-5), and also the Shellsol® products from Shell, Caromax® (e.g., Caromax® 18) from Petrochem Carless, and Hydrosol from DHC (e.g., as Hydrosol® A 170). Hydrocarbon mixtures composed of paraffins, cycloparaffins, and aromatics are also available commercially under the designations Kristalloel (for example, Kristalloel 30, boiling range about 158-198° C., or Kristalloel 60: CAS No. 64742-82-1), white spirit (for example, likewise CAS No. 64742-82-1), or solvent naphtha (light: boiling range about 155-180° C., heavy: boiling range about 225-300° C.). The aromatics content of such hydrocarbon mixtures is generally more than 90 wt %, specifically more than 95, more specifically more than 98, and very specifically more than 99 wt %. It may be advisable to use hydrocarbon mixtures having a particularly reduced naphthalene content.

(Cyclo)aliphatic hydrocarbons are, for example, decalin, alkylated decalin, and isomer mixtures of linear or branched alkanes and/or cycloalkanes.

The aliphatic hydrocarbon content is generally less than 5, specifically less than 2.5, and more specifically less than 1 wt %.

Esters are, for example, n-butyl acetate, ethyl acetate, 1-methoxyprop-2-yl acetate, and 2-methoxyethyl acetate.

Ethers are, for example, THF, dioxane, and the dimethyl, diethyl, or di-n-butyl ethers of ethylene glycol, diethylene glycol, triethylene glycol, propylene glycol, dipropylene glycol, or tripropylene glycol.

Ketones are, for example, acetone, diethyl ketone, ethyl methyl ketone, isobutyl methyl ketone, methyl amyl ketone, and tert-butyl methyl ketone.

In one or more embodiments, the solvents are n-butyl acetate, ethyl acetate, 1-methoxyprop-2-yl acetate, 2-methoxyethyl acetate, and also mixtures thereof, especially with the aromatic hydrocarbon mixtures recited above, more particularly xylene and Solvesso® 100.

Such mixtures may be made in a volume ratio of 5:1 to 1:5, specifically in a volume ratio of 4:1 to 1:4, more specifically in a volume ratio of 3:1 to 1:3, and very specifically in a volume ratio of 2:1 to 1:2.

In one or more embodiments, examples are butyl acetate/xylene, 1:1 methoxypropyl acetate/xylene, 1:1 butyl acetate/solvent naphtha 100, 1:2 butyl acetate/Solvesso® 100, and 3:1 Kristalloel 30/Shellsol® A.

In one or more embodiments, the solvents are selected from butyl acetate, 1-methoxyprop-2-yl acetate, methyl amyl ketone, xylene, and Solvesso® 100.

In general it is necessary and preferable for the reaction to be carried out in the presence of at least one stabilizer to counter radical polymerization of component (A), this stabilizer being specifically hydroquinone monomethyl ether and/or phenothiazine. It is also possible, though, for other stabilizers known for the stabilization of (meth)acrylates with respect to radical polymerization to be used.

The first reaction step is at an end when the lactone (B) has undergone substantial reaction, specifically to an extent of at least 90%, more specifically at least 95, very specifically at least 97, and more particularly at least 98%.

It is possible for unreacted lactone (B) and solvent optionally used, and water, if the water content is >1000 ppm, to be removed from the reaction mixture, specifically by distillation, though in a specific embodiment the reaction mixture obtained from the first step is used directly in the second step, the reaction with component (D).

It is possible to terminate the reaction from the first step specifically by cooling. In this form the reaction mixture can be stored and can then be used at a later point in time, in the second step.

In the second reaction step, the reaction mixture obtained from the first step is then reacted with component (D).

The second reaction step is carried out in a stoichiometry of 1.2:1 to 1:1.2 in terms of hydroxyl groups in the reaction product from the first step to isocyanate groups in component (D), specifically 1.1:1 to 1:1.1, more specifically 1.05:1 to 1:1.05, and very specifically 1:1.

The reaction in the second step takes place specifically at 40 to 100° C., more specifically 50 to 90, very specifically at 60 to 80° C.

For this step, the reaction mixture obtained from the first reaction step is brought to the desired temperature and component (D) is introduced in two or more portions or, specifically, in one portion.

In one or more embodiments, the catalyst (C), present in the reaction mixture from the reaction in the first step, is sufficient to catalyze the reaction between isocyanate groups and hydroxyl groups as well. Should this not be the case, then further catalyst (C) may be metered in subsequently. This may be the same catalyst (C) as in the first step, or a different one; specifically, the same catalyst.

The reaction is continued until the NCO value has dropped to below 1 wt %, specifically below 0.5 wt %, more specifically below 0.3, very specifically below 0.2, and more particularly below 0.1 wt %.

If the reaction has been carried out in the presence of a solvent, this solvent can now be separated off, specifically by distillation.

It is possible, although generally not necessary, for the catalyst to be removed from the resulting reaction mixture.

Its removal may take place, for example, by washing or filtration.

For this purpose the reaction mixture is neutralized in a washing apparatus with a 5-25, specifically 5-20, more specifically 5-15 wt % strength aqueous solution of a base, such as sodium hydroxide, potassium hydroxide, sodium hydrogencarbonate, sodium carbonate, potassium hydrogencarbonate, calcium hydroxide, aqueous ammonia, or potassium carbonate, for example, which may optionally have been admixed with 5-15 wt % of sodium chloride, potassium chloride, ammonium chloride, or ammonium sulfate, neutralization taking place specifically with aqueous sodium hydroxide solution or aqueous sodium hydroxide/sodium chloride solution.

Washing may be carried out, for example, in a stirred tank or in other conventional apparatus, such as in a column or mixer-settler apparatus, for example.

The organic phase from the initial wash is then treated with water or with a 5-30 wt %, specifically 5-20, more specifically 5-15 wt % strength solution of sodium chloride, potassium chloride, ammonium chloride, sodium sulfate, or ammonium sulfate, specifically sodium chloride solution.

It is also possible, however, for traces of catalyst to be removed from the reaction mixture by filtering it over activated carbon, aluminum oxide, silica, or ion exchangers.

The urethane (meth)acrylates of the invention or the product obtained in accordance with the method of the invention can be used in a conventional way in radiation-curable coating materials and has the advantage that in the product of the first stage, the distribution of the lactone units (B) is more uniform than in accordance with the processes from the prior art. A consequence of this is that the coating materials which comprise a product obtained by the process of the invention exhibit a higher flexibility.

Likewise provided by the present invention, accordingly, is the use of urethane (meth)acrylates obtained by the process of the invention in radiation-curable coating materials.

The urethane (meth)acrylates of the invention may be used as sole binder or, specifically, in combination with at least one further radically polymerizable compound.

Hence the present invention further provides radiation-curable coating materials comprising at least one urethane (meth)acrylate of the invention and optionally at least one radically polymerizable compound and also optionally at least one photoinitiator.

Radically polymerizable groups are, for example, specifically (meth)acrylate groups and more specifically acrylate groups.

The radically polymerizable compounds are specifically multifunctional (compound having more than one radically polymerizable double bond) polymerizable compounds.

The polymerizable compounds are specifically selected from the group consisting of multifunctional (meth)acrylates, urethane (meth)acrylates, epoxy (meth)acrylates, and carbonate (meth)acrylates.

(Meth)acrylic acid stands in this specification for methacrylic acid and acrylic acid, specifically for acrylic acid.

Multifunctional polymerizable compounds are specifically multifunctional (meth)acrylates which carry at least 2, specifically 2-10, more specifically 3-6, and very specifically 3-4 (meth)acrylate groups, specifically acrylate groups.

Examples of multifunctional polymerizable compounds are ethylene glycol diacrylate, 1,2-propanediol diacrylate, 1,3-propanediol diacrylate, 1,4-butanediol diacrylate, 1,3-butanediol diacrylate, 1,5-pentanediol diacrylate, 1,6-hexanediol diacrylate, 1,8-octanediol diacrylate, neopentyl glycol diacrylate, 1,1-, 1,2-, 1,3-, and 1,4-cyclohexanedimethanol diacrylate, 1,2-, 1,3- or 1,4-cyclohexanediol diacrylate, dipropylene glycol diacrylate, tripropylene glycol diacrylate, trimethylolpropane triacrylate, ditrimethylolpropane tetraacrylate, dipentaerythritol penta- or hexaacrylate, pentaerythritol tri- or tetraacrylate, glycerol di- or triacrylate, and also di- and polyacrylates of sugar alcohols, such as sorbitol, mannitol, diglycerol, threitol, erythritol, adonitol (ribitol), arabitol (lyxitol), xylitol, dulcitol (galactitol), maltitol or isomalt, or of polyester polyols, polyetherols, polyTHF having a molar mass of between 162 and 2000, poly-1,3-propanediol having a molar mass of between 134 and 1178, polyethylene glycol having a molar mass of between 106 and 898, and also epoxy (meth)acrylates, polyester (meth)acrylates, polyether (meth)acrylates, urethane (meth)acrylates or polycarbonate (meth)acrylates, which optionally may also have been modified with one or more amines.

Further examples are (meth)acrylates of compounds of formula (VIIIa) to (VIIId)

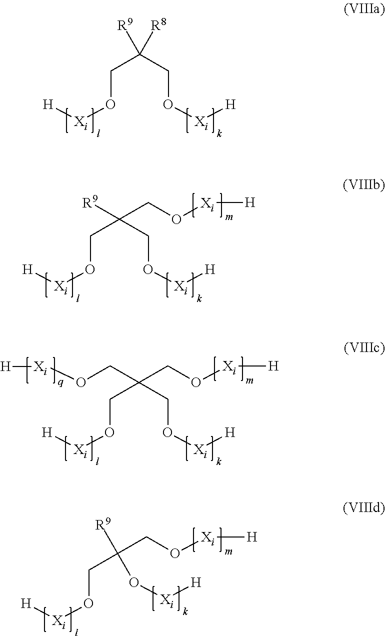

in which
R⁸ and R⁹ independently of one another are hydrogen or are C₁-C₁₈ alkyl which is optionally substituted by aryl, alkyl, aryloxy, alkyloxy, heteroatoms and/or heterocycles,
k, l, m, and q independently of one another are each an integer from 1 to 10, specifically 1 to 5, and more specifically 1 to 3, and
each Xᵢ for i=1 to k, 1 to l, 1 to m, and 1 to q can be selected independently of one another from the group —CH₂—CH₂—O—, —CH₂—CH(CH₃)—O—, —CH(CH₃)—CH₂—O—, —CH₂—C(CH₃)₂—O—, —C(CH₃)₂—CH₂—O—, —CH₂-CHVin-O—, —CHVin-CH₂—O—, —CH₂—CHPh-O—, and —CHPh-CH₂—O—, specifically from the group —CH₂—CH₂—O—, —CH₂—CH(CH₃)—O—, and —CH(CH₃)—CH₂—O—, and more specifically —CH₂—CH₂—O—,
in which Ph is phenyl and Vin is vinyl.
C₁-C₁₈ alkyl therein, optionally substituted by aryl, alkyl, aryloxy, alkyloxy, heteroatoms and/or heterocycles, is for example methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, hexadecyl, octadecyl, 1,1-dimethylpropyl, 1,1-dimethylbutyl, 1,1,3,3-tetramethylbutyl, specifically methyl, ethyl or n-propyl, more specifically methyl or ethyl.

These are specifically (meth)acrylates of singly to 20-tuply and more specifically triply to decuply ethoxylated, propoxylated or mixedly ethoxylated and propoxylated, and in particular exclusively ethoxylated, neopentyl glycol, trimethylolpropane, trimethylolethane or pentaerythritol.

In one or more embodiments, the multifunctional polymerizable compounds are 1,2-propanediol diacrylate, 1,3-propanediol diacrylate, dipropylene glycol diacrylate, tripropylene glycol diacrylate, trimethylolpropane triacrylate, ditrimethylol tetraacrylate, and dipentaerythritol hexaacrylate, polyester polyol acrylates, polyetherol acrylates, and triacrylate of singly to vigintuply alkoxylated, more specifically singly to 20-tuply ethoxylated trimethylolpropane, singly to 20-tuply propoxylated glycerol or singly to 20-tuply ethoxylated and/or propoxylated pentaerythritol.

In one specific embodiment, epoxy (meth)acrylates are used as multifunctional polymerizable compounds in print varnishes.

In one or more embodiments, the multifunctional polymerizable compounds are trimethylolpropane triacrylate and triacrylate of singly to vigintuply ethoxylated trimethylolpropane, triacrylate of singly to 20-tuply propoxylated glycerol or tetraacrylate of singly to 20-tuply ethoxylated and/or propoxylated pentaerythritol.

Further constituents may also be polyalcohols with full or partial esterification with (meth)acrylic acid.

Examples of such polyalcohols are at least divalent polyols, polyetherols or polyesterols, or polyacrylate polyols, having an average OH functionality of at least 2, specifically at least 3, more specifically at least 4, and very specifically 4 to 20.

Polyetherols, in addition to the alkoxylated polyols, may also be polyethylene glycol having a molar mass of between 106 and 2000, polypropylene glycol having a molar weight of between 134 and 2000, polyTHF having a molar weight of between 162 and 2000, or poly-1,3-propanediol having a molar weight of between 134 and 400.

Polyester polyols are known for example from Ullmanns Encyklopädie der technischen Chemie, 4th edition, volume 19, pp. 62 to 65. In one or more embodiments, polyester polyols obtained by reacting dihydric alcohols with dibasic carboxylic acids are used. In lieu of the free polycarboxylic acids it is also possible to use the corresponding polycarboxylic anhydrides or corresponding polycarboxylic esters of lower alcohols or mixtures thereof to prepare the polyester polyols. The polycarboxylic acids may be aliphatic, cycloaliphatic, araliphatic, aromatic or heterocyclic and may optionally be substituted, by halogen atoms for example, and/or unsaturated. Examples thereof that may be mentioned include the following:

oxalic acid, maleic acid, fumaric acid, succinic acid, glutaric acid, adipic acid, sebacic acid, dodecanedioic acid, o-phthalic acid, isophthalic acid, terephthalic acid, trimellitic acid, azelaic acid, 1,4-cyclohexanedicarboxylic acid or tetrahydrophthalic acid, suberic acid, azelaic acid, phthalic anhydride, tetrahydrophthalic anhydride, hexahydrophthalic anhydride, tetrachlorophthalic anhydride, endomethylenetetrahydrophthalic anhydride, glutaric anhydride, maleic anhydride, dimeric fatty acids, their isomers and hydrogenation products, and also esterifiable derivatives, such as anhydrides or dialkyl esters, C₁-C₄-alkyl esters for example, specifically methyl, ethyl or n-butyl esters, of said acids are used. In one or more embodiments, dicarboxylic acids of the general formula HOOC—(CH₂)_{y}—COOH, y being a number from 1 to 20, specifically an even number from 2 to 20; more specifically succinic acid, adipic acid, sebacic acid, and dodecanedicarboxylic acid are used.

Suitable polyhydric alcohols for preparing the polyesterols include 1,2-propanediol, ethylene glycol, 2,2-dimethyl-1,2-ethanediol, 1,3-propanediol, 1,2-butanediol, 1,3-butanediol, 1,4-butanediol, 3-methylpentane-1,5-diol, 2-ethylhexane-1,3-diol, 2,4-diethyloctane-1,3-diol, 1,6-hexanediol, polyethylene glycol having a molar mass between 106 and 2000, polypropylene glycol having a molar weight between 134 and 2000, polyTHF having a molar weight between 162 and 2000, poly-1,3-propanediol having a molar weight between 134 and 400, neopentyl glycol, neopentyl glycol hydroxypivalate, 2-ethyl-1,3-propanediol, 2-methyl-1,3-propanediol, 2,2-bis(4-hydroxycyclohexyl)propane, 1,1-, 1,2-, 1,3-, and 1,4-cyclohexanedimethanol, 1,2-, 1,3- or 1,4-cyclohexanediol, trimethylolbutane, trimethylolpropane, trimethylolethane, neopentyl glycol, pentaerythritol, glycerol, ditrimethylolpropane, dipentaerythritol, sorbitol, mannitol, diglycerol, threitol, erythritol, adonitol (ribitol), arabitol (lyxitol), xylitol, dulcitol (galactitol), maltitol or isomalt, which optionally may have been alkoxylated as described above.

In one or more embodiments, the alcohols are those of the general formula HO—(CH₂)ₓ—OH, x being a number from 1 to 20, specifically an even number from 2 to 20. In one or more embodiments, ethylene glycol, butane-1,4-diol, hexane-1,6-diol, octane-1,8-diol, and dodecane-1,12-diol are used. In specific embodiments, neopentyl glycol is used.

Also suitable are lactone-based polyesterdiols, which are homopolymers or copolymers of lactones, specifically hydroxyl-terminated adducts of lactones with suitable difunctional starter molecules. Suitable lactones include, specifically, those deriving from compounds of the general formula HO—(CH₂)_{z}—COOH, z being a number from 1 to 20 and it being possible for an H atom of a methylene unit also to have been substituted by a C₁ to C₄ alkyl radical. Examples are ε-caprolactone, β-propiolactone, gamma-butyrolactone and/or methyl-ε-caprolactone, 4-hydroxybenzoic acid, 6-hydroxy-2-naphthoic acid or pivalolactone, and mixtures thereof. Examples of suitable starter components are the low molecular mass dihydric alcohols specified above as a synthesis component for the polyester polyols. In one or more specific embodiments, the corresponding polymers of ε-caprolactone are used. Lower polyesterdiols or polyetherdiols as well can be used as starters for preparing the lactone polymers. In lieu of the polymers of lactones it is also possible to use the corresponding, chemically equivalent polycondensates of the hydroxycarboxylic acids corresponding to the lactones.

Also suitable, furthermore, are polycarbonate diols, such as may be obtained, for example, by reacting phosgene with an excess of the low molecular weight alcohols specified as synthesis components for the polyester polyols.

The multifunctional polymerizable compound may also comprise urethane (meth)acrylates, epoxy (meth)acrylates or carbonate (meth)acrylates.

Urethane (meth)acrylates are obtainable for example by reacting polyisocyanates with hydroxyalkyl (meth)acrylates and optionally chain extenders such as diols, polyols, diamines, polyamines, dithiols or polythiols. Urethane (meth)acrylates which can be dispersed in water without addition of emulsifiers additionally comprise ionic and/or nonionic hydrophilic groups, which are introduced into the urethane by means of synthesis components such as hydroxycarboxylic acids, for example.

Urethane (meth)acrylates of this kind comprise as synthesis components substantially:
(1) at least one organic aliphatic, aromatic or cycloaliphatic, specifically aliphatic or cycloaliphatic di- or polyisocyanate,
(2) at least one compound having at least one isocyanate-reactive group and at least one radically polymerizable unsaturated group, and
(3) optionally, at least one compound having at least two isocyanate-reactive groups.

The urethane (meth)acrylates specifically have a number-average molar weight Mₙ of 500 to 20 000, in particular of 500 to 10 000 and more specifically 600 to 3000 g/mol (determined by gel permeation chromatography using tetrahydrofuran and polystyrene as standard).

The urethane (meth)acrylates specifically have a (meth)acrylic group content of 1 to 5, more specifically of 2 to 4, mol per 1000 g of urethane (meth)acrylate.

Epoxy (meth)acrylates are obtainable by reacting epoxides with (meth)acrylic acid. Examples of suitable epoxides include epoxidized olefins, aromatic glycidyl ethers or aliphatic glycidyl ethers, specifically those of aromatic or aliphatic glycidyl ethers.

Examples of possible epoxidized olefins include ethylene oxide, propylene oxide, isobutylene oxide, 1-butene oxide, 2-butene oxide, vinyloxirane, styrene oxide or epichlorohydrin, preference being given to ethylene oxide, propylene oxide, isobutylene oxide, vinyloxirane, styrene oxide or epichlorohydrin, particular preference to ethylene oxide, propylene oxide or epichlorohydrin, and very particular preference to ethylene oxide and epichlorohydrin.

Aromatic glycidyl ethers are, for example, bisphenol A diglycidyl ether, bisphenol F diglycidyl ether, bisphenol B diglycidyl ether, bisphenol S diglycidyl ether, hydroquinone diglycidyl ether, alkylation products of phenol/dicyclopentadiene, e.g., 2,5-bis[(2,3-epoxypropoxy)phenyl]octahydro-4,7-methano-5H-indene (CAS No. [13446-85-0]), tris[4-(2,3-epoxypropoxy)phenyl]methane isomers (CAS No. [66072-39-7]), phenol-based epoxy novolaks (CAS No. [9003-35-4]), and cresol-based epoxy novolaks (CAS No. [37382-79-9]).

Examples of aliphatic glycidyl ethers include 1,4-butanediol diglycidyl ether, 1,6-hexanediol diglycidyl ether, trimethylolpropane triglycidyl ether, pentaerythritol tetraglycidyl ether, 1,1,2,2-tetrakis[4-(2,3-epoxypropoxy)phenyl]ethane (CAS No. [27043-37-4]), diglycidyl ether of polypropylene glycol (α,ω-bis(2,3-epoxypropoxy)poly(oxypropylene), CAS No. [16096-30-3]) and of hydrogenated bisphenol A (2,2-bis[4-(2,3-epoxypropoxy)cyclohexyl]propane, CAS No. [13410-58-7]).

The epoxy (meth)acrylates specifically have a number-average molar weight Mₙ of 200 to 20 000, more specifically of 200 to 10 000 g/mol, and very specifically of 250 to 3000 g/mol; the amount of (meth)acrylic groups is specifically 1 to 5, more specifically 2 to 4, per 1000 g of epoxy (meth)acrylate (determined by gel permeation chromatography using polystyrene as standard and tetrahydrofuran as eluent).

Carbonate (meth)acrylates comprise on average specifically 1 to 5, especially 2 to 4, more specifically 2 to 3 (meth)acrylic groups, and very specifically 2 (meth)acrylic groups.

The number-average molecular weight Mₙ of the carbonate (meth)acrylates is specifically less than 3000 g/mol, more specifically less than 1500 g/mol, very specifically less than 800 g/mol (determined by gel permeation chromatography using polystyrene as standard, tetrahydrofuran as solvent).

The carbonate (meth)acrylates are obtainable in a simple manner by transesterifying carbonic esters with polyhydric, specifically dihydric, alcohols (diols, hexanediol for example) and subsequently esterifying the free OH groups with (meth)acrylic acid, or else by transesterification with (meth)acrylic esters, as described for example in EP-A 92 269. They are also obtainable by reacting phosgene, urea derivatives with polyhydric, e.g., dihydric, alcohols.

Also conceivable are (meth)acrylates of polycarbonate polyols, such as the reaction product of one of the aforementioned diols or polyols and a carbonic ester and also a hydroxyl-containing (meth)acrylate.

Examples of suitable carbonic esters include ethylene carbonate, 1,2- or 1,3-propylene carbonate, dimethyl carbonate, diethyl carbonate or dibutyl carbonate.

Examples of suitable hydroxyl-containing (meth)acrylates are 2-hydroxyethyl (meth)acrylate, 2- or 3-hydroxypropyl (meth)acrylate, 1,4-butanediol mono(meth)acrylate, neopentyl glycol mono(meth)acrylate, glyceryl mono- and di(meth)acrylate, trimethylolpropane mono- and di(meth)acrylate, and pentaerythritol mono-, di-, and tri(meth)acrylate.

In one or more embodiments, the carbonate (meth)acrylates are those of the formula:

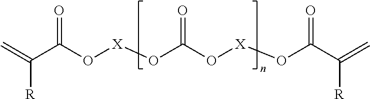

wherein R is H or CH₃, X is a C₂-C₁₈ alkylene group, and n is an integer from 1 to 5, specifically 1 to 3.
R is specifically H and X is specifically C₂ to C₁₀ alkylene, examples being 1,2-ethylene, 1,2-propylene, 1,3-propylene, 1,4-butylene, and 1,6-hexylene, more specifically C₄ to C₈ alkylene. With very particular preference X is C₆ alkylene.

The carbonate (meth)acrylates are specifically aliphatic carbonate (meth)acrylates.

Among the multifunctional polymerizable compounds, urethane (meth)acrylates are particularly preferred.

In one or more embodiments, at least one photoinitiator is added to the coating materials of the invention.

Photoinitiators may be, for example, photoinitiators known to the skilled person, examples being those specified in “Advances in Polymer Science”, Volume 14, Springer Berlin 1974 or in K. K. Dietliker, Chemistry and Technology of UV and EB Formulation for Coatings, Inks and Paints, Volume 3; Photoinitiators for Free Radical and Cationic Polymerization, P. K. T. Oldring (Eds), SITA Technology Ltd, London.

Suitability is possessed, for example, by mono- or bisacylphosphine oxides, as described for example in EP-A 7 508, EP-A 57 474, DE-A 196 18 720, EP-A 495 751 or EP-A 615 980, examples being 2,4,6-trimethylbenzoyldiphenylphosphine oxide (Lucirin® TPO from BASF SE), ethyl 2,4,6-trimethylbenzoylphenylphosphinate (Lucirin® TPO L from BASF SE), bis(2,4,6-trimethylbenzoyl)phenylphosphine oxide (Irgacure® 819 from BASF SE), benzophenones, hydroxyacetophenones, phenylglyoxylic acid and its derivatives, or mixtures of these photoinitiators. Examples that may be mentioned include benzophenone, acetophenone, acetonaphthoquinone, methyl ethyl ketone, valerophenone, hexanophenone, α-phenylbutyrophenone, p-morpholinopropiophenone, dibenzosuberone, 4-morpholinobenzophenone, 4-morpholinodeoxybenzoin, p-diacetylbenzene, 4-aminobenzophenone, 4′-methoxyacetophenone, 3-methylanthraquinone, tert-butylanthraquinone, anthraquinonecarboxylic esters, benzaldehyde, α-tetralone, 9-acetylphenanthrene, 2-acetylphenanthrene, 10-thioxanthone, 3-acetylphenanthrene, 3-acetylindole, 9-fluorenone, 1-indanone, 1,3,4-triacetylbenzene, thioxanthen-9-one, xanthen-9-one, 2,4-dimethylthioxanthone, 2,4-diethylthioxanthone, 2,4-diisopropylthioxanthone, 2,4-dichlorothioxanthone, benzoin, benzoin isobutyl ether, chloroxanthenone, benzoin tetrahydropyranyl ether, benzoin methyl ether, benzoin ethyl ether, benzoin butyl ether, benzoin isopropyl ether, 7H-benzoin methyl ether, benz[de]anthracen-7-one, 1-naphthaldehyde, 4,4′-bis(dimethylamino)benzophenone, 4-phenylbenzophenone, 4-chlorobenzophenone, Michler's ketone, 1-acetonaphthone, 2-acetonaphthone, 1-benzoylcyclohexan-1-ol, 2-hydroxy-2,2-dimethylacetophenone, 2,2-dimethoxy-2-phenylacetophenone, 2,2-diethoxy-2-phenylacetophenone, 1,1-dichloroacetophenone, 1-hydroxyacetophenone, acetophenone dimethyl ketal, o-methoxybenzophenone, 2-hydroxy-1-[4-[[4-(2-hydroxy-2-methylpropanoyl)phenyl]methyl]phenyl]-2-methylpropan-1-one, 2-benzyl-2-dimethylamino-4′-morpholinobutyrophenone, 2-(dimethylamino)-1-(4-morpholinophenyl)-2-(p-tolylmethyl)butan-1-one, 2-methyl-1-(4-methylthiophenyl)-2-morpholinopropan-1-one, triphenylphosphine, tri-o-tolylphosphine, benz[a]anthracene-7,12-dione, 2,2-diethoxyacetophenone, benzil ketals, such as benzil dimethyl ketal, anthraquinones such as 2-methylanthraquinone, 2-ethylanthraquinone, 2-tert-butylanthraquinone, 1-chloroanthraquinone, 2-amylanthraquinone, and 2,3-butanedione.

Likewise conceivable as photoinitiators are polymeric photoinitiators such as, for example, the diester of carboxymethoxybenzophenone with polytetramethylene glycols of different molar weights, specifically 200 to 250 g/mol (CAS 515136-48-8), and also CAS 1246194-73-9, CAS 813452-37-8, CAS 71512-90-8, CAS 886463-10-1, or further polymeric benzophenone derivatives, of the kind available commercially, for example, under the trade name Omnipol® BP from IGM Resins B.V., Waalwijk, The Netherlands, or Genopol® BP1 from Rahn AG, Switzerland. Also conceivable, furthermore, are polymeric thioxanthones, examples being the diester of carboxymethoxythioxanthones with polytetramethylene glycols of various molar weights, of the kind available commercially, for example, under the trade name Omnipol® TX from IGM Resins B.V., Waalwijk, The Netherlands. Also conceivable, furthermore, are polymeric α-amino ketones, examples being the diester of carboxyethoxythioxanthones with polyethylene glycols of various molar weights, of the kind available commercially, for example, under the trade name Omnipol® 910 or Omnipol® 9210 from IGM Resins B.V., Waalwijk, The Netherlands.

One embodiment uses, as photoinitiators, silsesquioxane compounds having at least one initiating group, of the kind described in WO 2010/063612 A1, particularly from page 2, line 21 to page 43, line 9 therein, hereby incorporated by reference as part of the present disclosure content, specifically from page 2, line 21 to page 30, line 5, and also the compounds described in the examples of WO 2010/063612 A1.

Also suitable are nonyellowing or low-yellowing photoinitiators of the phenylglyoxalic ester type, as described in DE-A 198 26 712, DE-A 199 13 353 or WO 98/33761, and silsesquioxane compounds.

Preference among these photoinitiators is given to 2,4,6-trimethylbenzoyldiphenylphosphine oxide, ethyl 2,4,6-trimethylbenzoylphenylphosphinate, bis(2,4,6-trimethylbenzoyl)phenylphosphine oxide, 2-benzyl-2-dimethylamino-4′-morpholinobutyrophenone, 2-(dimethylamino)-1-(4-morpholinophenyl)-2-(p-tolylmethyl)butan-1-one, 2-hydroxy-1-[4-[[4-(2-hydroxy-2-methyl-propanoyl)phenyl]methyl]phenyl]-2-methylpropan-1-one, and also the above-described polymeric thioxanthone and benzophenone derivatives, and also those described in WO 2010/063612 A1.

As further typical additives to the coating materials it is possible for example to use dispersants, waxes, stabilizers, sensitizers, fillers, defoamers, colorants, antistatic agents, thickeners, surface-active agents such as flow control agents, slip aids or adhesion promoters.

Suitable fillers comprise silicates, examples being silicates obtainable by hydrolysis of silicon tetrachloride, such as Aerosil® from Degussa, siliceous earth, talc, aluminum silicates, magnesium silicates, calcium carbonates, etc.

Recited below are examples of particularly suitable pigments which may be added to the coating materials of the invention.

### Organic Pigments:

Monoazo pigments: C.I. Pigment Brown 25; C.I. Pigment Orange 5, 13, 36 and 67;
   C.I. Pigment Red 1, 2, 3, 5, 8, 9, 12, 17, 22, 23, 31, 48:1, 48:2, 48:3, 48:4, 49, 49:1, 52:1, 52:2, 53, 53:1, 53:3, 57:1, 63, 112, 146, 170, 184, 210, 245 and 251; C.I. Pigment Yellow 1, 3, 73, 74, 65, 97, 151 and 183;
Disazo pigments: C.I. Pigment Orange 16, 34 and 44; C.I. Pigment Red 144, 166, 214 and 242; C.I. Pigment Yellow 12, 13, 14, 16, 17, 81, 83, 106, 113, 126, 127, 155, 174, 176 and 188;
Anthanthrone pigments: C.I. Pigment Red 168 (C.I. Vat Orange 3);
Anthraquinone pigments: C.I. Pigment Yellow 147 and 177; C.I. Pigment Violet 31;
Anthrapyrimidine pigments: C.I. Pigment Yellow 108 (C.I. Vat Yellow 20);
Quinacridone pigments: C.I. Pigment Red 122, 202 and 206; C.I. Pigment Violet 19;
Quinophthalone pigments: C.I. Pigment Yellow 138;
Dioxazine pigments: C.I. Pigment Violet 23 and 37;
Flavanthrone pigments: C.I. Pigment Yellow 24 (C.I. Vat Yellow 1);
Indanthrone pigments: C.I. Pigment Blue 60 (C.I. Vat Blue 4) and 64 (C.I. Vat Blue 6);
Isoindoline pigments: C.I. Pigment Orange 69; C.I. Pigment Red 260; C.I. Pigment Yellow 139 and 185;
Isoindolinone pigments: C.I. Pigment Orange 61; C.I. Pigment Red 257 and 260; C.I. Pigment Yellow 109, 110, 173 and 185;
Isoviolanthrone pigments: C.I. Pigment Violet 31 (C.I. Vat Violet 1);
Metal complex pigments: C.I. Pigment Yellow 117, 150 and 153; C.I. Pigment Green 8;
Perinone pigments: C.I. Pigment Orange 43 (C.I. Vat Orange 7); C.I. Pigment Red 194 (C.I. Vat Red 15);
Perylene pigments: C.I. Pigment Black 31 and 32; C.I. Pigment Red 123, 149, 178, 179 (C.I. Vat Red 23), 190 (C.I. Vat Red 29) and 224; C.I. Pigment Violet 29;
Phthalocyanine pigments: C.I. Pigment Blue 15, 15:1, 15:2, 15:3, 15:4, 15:6 and 16; C.I. Pigment Green 7 and 36;
Pyranthrone pigments: C.I. Pigment Orange 51; C.I. Pigment Red 216 (C.I. Vat Orange 4);
Thioindigo pigments: C.I. Pigment Red 88 and 181 (C.I. Vat Red 1); C.I. Pigment Violet 38 (C.I. Vat Violet 3);
Triarylcarbonium pigments: C.I. Pigment Blue 1, 61 and 62; C.I. Pigment Green 1; C.I. Pigment Red 81, 81:1 and 169; C.I. Pigment Violet 1, 2, 3 and 27;
   C.I. Pigment Black 1 (aniline black);
   C.I. Pigment Yellow 101 (aldazine yellow);
   C.I. Pigment Brown 22.

### Inorganic Pigments:

White pigments: titanium dioxide (C.I. Pigment White 6), zinc white, pigmented zinc oxide, barium sulfate, zinc sulfide, lithopones; lead white; calcium carbonate;
Black pigments: iron oxide black (C.I. Pigment Black 11), iron-manganese black, spinel black (C.I. Pigment Black 27); carbon black (C.I. Pigment Black 7);
Color pigments: chromium oxide, chromium oxide hydrate green; chromium green (C.I. Pigment Green 48); cobalt green (C.I. Pigment Green 50); ultramarine green; cobalt blue (C.I. Pigment Blue 28 and 36); ultramarine blue; iron blue (C.I. Pigment Blue 27); manganese blue; ultramarine violet; cobalt and manganese violet; iron oxide red (C.I. Pigment Red 101); cadmium sulfoselenide (C.I. Pigment Red 108); molybdate red (C.I. Pigment Red 104); ultramarine red;

Iron oxide brown, mixed brown, spinel and corundum phases (C.I. Pigment Brown 24, 29 and 31), chromium orange;

Iron oxide yellow (C.I. Pigment Yellow 42); nickel titanium yellow (C.I. Pigment Yellow 53; C.I. Pigment Yellow 157 and 164); chromium titanium yellow; cadmium sulfide and cadmium zinc sulfide (C.I. Pigment Yellow 37 and 35); chromium yellow (C.I. Pigment Yellow 34), zinc yellow, alkaline earth metal chromates; Naples yellow; bismuth vanadate (C.I. Pigment Yellow 184);
Interference pigments: metallic effect pigments based on coated metal platelets; pearlescent pigments based on metal oxide coated mica platelets; liquid crystal pigments.

In one or more embodiments, pigments in this context are monoazo pigments (especially laked BONS pigments, Naphthol AS pigments), disazo pigments (especially diary) yellow pigments, bisacetoacetanilide pigments, disazopyrazolone pigments), quinacridone pigments, quinophthalone pigments, perinone pigments, phthalocyanine pigments, triarylcarbonium pigments (alkali blue pigments, laked rhodamines, dye salts with complex anions), isoindoline pigments, white pigments, and carbon blacks.

Examples of particular pigments are specifically: carbon black, titanium dioxide, C.I. Pigment Yellow 138, C.I. Pigment Red 122 and 146, C.I. Pigment Violet 19, C.I. Pigment Blue 15:3 and 15:4, C.I. Pigment Black 7, C.I. Pigment Orange 5, 38 and 43, and C.I. Pigment Green 7.

Suitable stabilizers comprise typical UV absorbers such as oxanilides, triazines, and benzotriazole (the latter obtainable as Tinuvin® grades from BASF), and benzophenones. They can be employed alone or together with suitable free-radical scavengers, examples being sterically hindered amines such as 2,2,6,6-tetramethylpiperidine, 2,6-di-tert-butylpiperidine or derivatives thereof, e.g., bis(2,2,6,6-tetramethyl-4-piperidyl) sebacate, or quinone methide (such as Irgastab® UV22). Stabilizers are used usually in amounts of 0.1 to 0.5 wt % of the active ingredient component, based on the preparation.

The coating materials may also be used as printing-inks. A further aspect of the present invention is a process for printing sheetlike or three-dimensional, specifically sheetlike substrates by any desired printing process, using at least one printing-ink of the invention. In one variant of the printing process of the invention, at least one printing-ink of the invention is printed onto a substrate and then treated with actinic radiation, as for example UV radiation and/or electron beams, specifically UV radiation.

Printing processes in which the printing-inks of the invention can be used are specifically offset printing, letterpress, flexographic printing, gravure printing, screen printing, and inkjet printing, more specifically flexographic and offset printing.

In the so-called mechanical printing processes such as offset printing, letterpress, flexographic printing or gravure printing, the printing-ink is transferred to the print-receiving medium (printing stock) by contact with a printing form or printing plate which is inked with printing-ink. UV-curable printing-inks for these applications typically comprise reactive diluents, binders, colorants, initiators, and also, optionally, various additives. Binders serve to form the ink film and to anchor the constituents, such as pigments or fillers, for example, in the ink film. Depending on consistency, printing-inks for these applications typically comprise between 10 and 60 wt % of binder. Reactive diluents serve to adjust the processing viscosity.

Print varnishes are applied either to the printing stock, as a primer, or to the printing stock after the printing operation, as a coating. Print varnishes are used, for example, for protecting the printed image, for improving the adhesion of the printing-ink to the printing stock, or for esthetic purposes. Application is typically in-line or off-line by means of a varnishing unit on the printing machine.

Print varnishes contain no colorant, but apart from that generally have a similar composition to printing-inks, and are distinguished by the absence of the colorant.

Printing-inks for mechanical printing processes comprise what are called pasty printing-inks of high viscosity for offset printing and for letterpress, and also what are called liquid printing-inks, of comparatively low viscosity, for flexographic and gravure printing.

The inks of the invention can be used for example as ink-jet liquid and also for liquid toners for electrophotographic printing processes.

Optionally, if two or more printed layers of the printing-inks are applied one above another, it is possible for drying and/or radiation curing to take place after each printing operation.

Radiation curing takes place with high-energy light, UV light for example, or electron beams. Radiation curing may also take place at relatively high temperatures.

Examples of suitable radiation sources for the radiation cure are low-pressure mercury lamps, medium-pressure mercury lamps with high-pressure lamps, and fluorescent tubes, pulsed lamps, metal halide lamps, electronic flash units, with the result that radiation curing is possible without a photoinitiator, or excimer lamps and also UV LEDs. The radiation cure is accomplished by exposure to high-energy radiation, i.e., UV radiation, or daylight, specifically light in the wavelength range of λ=200 to 700 nm, more specifically λ=200 to 500 nm, and very specifically λ=250 to 420 nm, or by exposure to high-energy electrons (electron beams; 60 to 300 keV). Examples of radiation sources used include high-pressure mercury vapor lamps, lasers, pulsed lamps (flash light), halogen lamps, UV LEDs, or excimer lamps. The radiation dose normally sufficient for crosslinking in the case of UV curing is in the range from 30 to 3000 mJ/cm².

It will be appreciated that a number of radiation sources can also be used for the cure: two to four, for example.

These sources may also emit each in different wavelength ranges.

Irradiation can optionally also be carried out in the absence of oxygen, such as under an inert gas atmosphere. Suitable inert gases are specifically nitrogen, noble gases, carbon dioxide, or combustion gases.

The coating materials of the invention are suitable for coating substrates such as wood, paper, textile, leather, nonwoven, plastic surfaces, PVC, glass, ceramic, mineral building materials, such as molded cement blocks and fiber cement slabs, or metals or coated-metal substrates, specifically plastics or metals, more particularly in the form of foils, more specifically metals.

The coating materials may be used more particularly in primers, primer-surfacers, pigmented top coat materials, and clearcoat materials in the sectors of automotive refinish or large-vehicle finishing, and aircraft. Such coating materials are especially suitable for applications requiring particularly high reliability of application, outdoor weathering resistance, hardness, and flexibility, such as in automotive refinish and large-vehicle finishing.

The examples below are intended to illustrate the invention but not to limit it to these examples.

The % and ppm figures quoted in this specification pertain to wt-% and wt-ppm, unless otherwise indicated.

### EXAMPLES

### Comparative Example 1

323 parts of epsilon-caprolactone, 164 parts of hydroxyethyl acrylate, and 0.2 part of zinc ethylhexanoate (BorchiKat® 22 from OMG Borchers GmbH, Langenfeld, Germany) were heated at 105-110° C. for 11 hours, followed by cooling to 60° C. and addition of 187 parts of a diisocyanate based on H12-MDI (Desmodur® W from Bayer MaterialScience), and by reaction for a further 14 hours at 80-85° C. The isocyanate value had dropped to <0.1%. This gave a viscous, clear urethane acrylate having a viscosity of 27.5 Pas (measured using an Epprecht cone/plate viscosimeter (cone C) at 25° C.).

### Inventive Example 1

323 parts of epsilon-caprolactone, 164 parts of hydroxylethyl acrylate, and 0.2 part of zinc ethylhexanoate (BorchiKat® 22 from OMG Borchers GmbH, Langenfeld, Germany) were heated at 105-110° C. for 11 hours, followed by cooling to 60° C. and addition of 400 parts of an isocyanato acrylate (Laromer® LR9000), and by reaction for a further 12 hours at 80-85° C. The isocyanate value had dropped to <0.1%. This gave a viscous, clear urethane acrylate having a viscosity of 15 Pas (measured using an Epprecht cone/plate viscosimeter (cone C) at 25° C.).

### Inventive Example 2

323 parts of epsilon-caprolactone, 164 parts of hydroxyethyl acrylate, and 0.5 part of tetrabutyl orthotitanate were heated at 105-110° C. for 11 hours, followed by cooling to 60° C. and addition of 400 parts of an isocyanato acrylate (Laromer® LR9000), and by reaction for a further 20 hours at 80-85° C. The isocyanate value had dropped to <0.1%. This gave a viscous, clear urethane acrylate having a viscosity of 15.8 Pas (measured using an Epprecht cone/plate viscosimeter (cone C) at 25° C.).

### Inventive Example 3

323 parts of epsilon-caprolactone, 164 parts of hydroxyethyl acrylate, and 0.2 part of bismuth ethylhexanoate (BorchiKat® 24 from OMG Borchers GmbH, Langenfeld, Germany) were heated at 105-110° C. for 36 hours, followed by cooling to 60° C. and addition of 400 parts of an isocyanato acrylate (Laromer® LR9000), and by reaction for a further 12 hours at 80-85° C. The isocyanate value had dropped to <0.1%. This gave a viscous, clear urethane acrylate having a viscosity of 18 Pas (measured using an Epprecht cone/plate viscosimeter (cone C) at 25° C.).

### Example 4

### Production of the Coatings for Determining the Scratch Resistance

96 parts each of the urethane acrylates from inventive examples 1 to 3 and from comparative example 1 were mixed in each case with 4 parts of the photoinitiator Darocur® 1173 (2-hydroxy-2-methyl-1-phenyl-propan-1-one, photoinitiator from BASF SE), each applied to a black glass plate, using a four-way bar applicator (200 μm), and exposed using an IST UV exposure unit, and 1350 mJ/cm² exposure strength, in air.

The König pendulum damping was 18 s for comparative example 1 and 27 s for inventive example 1. High values stand for a high hardness.

The scratch resistance of the cured film was determined as follows:

The exposed films were scratched with 10 back-and-forth strokes under a load of 750 g, using a ScotchBrite® Fleece, and the difference in gloss before and after scratching was determined at a measurement angle of 60°. The gloss retention is the percentage value formed from gloss after scratching relative to gloss before scratching.

The gloss retention was as follows:

Comparative example 1: 52%
Inventive example 1: 94%
Inventive example 2: 91%
Inventive example 3: 93%

What is claimed is:

1. A urethane (meth)acrylate of the formula (I) 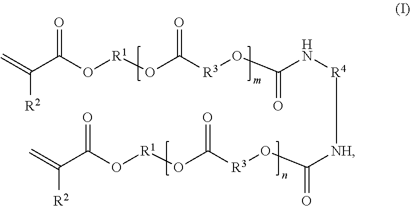
wherein
R¹ is a divalent alkylene radical having 2 to 12 carbon atoms and, optionally substituted with C₁ to C₄ alkyl groups and/or interrupted by one or more oxygen atoms,
R² in each case independently of any other is methyl or hydrogen,
R³ is a divalent alkylene radical having 1 to 12 carbon atoms and optionally substituted with C₁ to C₄ alkyl groups and/or interrupted by one or more oxygen atoms,
R⁴ is a divalent organic radical formed by conceptual abstraction of two isocyanate groups from a polyisocyanate (D) which comprises at least one hydroxyalkyl (meth)acrylate bonded via an allophanate group, and n and m independently of one another are positive numbers from 1 to 5.

2. The urethane (meth)acrylate according to claim 1, wherein R¹ is selected from the group consisting of 1,2-ethylene, 1,2- or 1,3-propylene, 1,2-, 1,3-, or 1,4-butylene, 1,1-dimethyl-1,2-ethylene, 1,2-dimethyl-1,2-ethylene, 1,5-pentylene, 1,6-hexylene, 1,8-octylene, 1,10-decylene, and 1,12-dodecylene.

3. The urethane (meth)acrylate according to claim 1, wherein R³ is selected from the group consisting of methylene, 1,2-ethylene, 1,2-propylene, 1,3-propylene, 1,2-butylene, 1,3-butylene, 1,4-butylene, 1,5-pentylene, 1,5-hexylene, 1,6-hexylene, 1,8-octylene, 1,10-decylene, 1,12-dodecylene, 2-oxa-1,4-butylene, 3-oxa-1,5-pentylene, or 3-oxa-1,5-hexylene.

4. The urethane (meth)acrylate according to claim 1, wherein the catalyst is a titanium compound, zinc compound, or bismuth compound.

5. A process for preparing the urethane (meth)acrylate according to claim 1, comprising:
in a first step reacting a hydroxyalkyl (meth)acrylate (A) of the formula 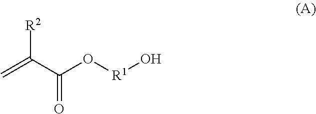
with a lactone (B) of the formula 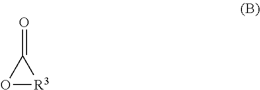
in the presence of at least one catalyst (C), selected from the group consisting of iron compounds, titanium compounds, aluminum compounds, zirconium compounds, manganese compounds, nickel compounds, zinc compounds, cobalt compounds, and bismuth compounds to provide a product; and,
in a further step, reacting the product from the first step with a polyisocyanate (D) which comprises at least one hydroxyalkyl (meth)acrylate bonded via an allophanate group.

6. The process according to claim 5, wherein the polyisocyanate (D) is obtained by reacting at least one (cyclo)aliphatic diisocyanate with at least one hydroxyalkyl (meth)acrylate in the presence of at least one catalyst able to accelerate the formation of allophanate groups.

7. The process according to claim 6, wherein the diisocyanate is selected from the group consisting of hexamethylene 1,6-diisocyanate, isophorone diisocyanate, and 4,4′- or 2,4′-di(isocyanatocyclohexyl)methane.

8. The process according to claim 6, wherein the at least one hydroxyalkyl (meth)acrylate used to prepared component (D) is selected from the group consisting of 2-hydroxyethyl (meth)acrylate, 2- or 3-hydroxypropyl (meth)acrylate, 1,4-butanediol mono(meth)acrylate, neopentyl glycol mono(meth)acrylate, 1,5-pentanediol mono(meth)acrylate, and 1,6-hexanediol mono(meth)acrylate.

9. The process according to claim 5, wherein the polyisocyanate (D) comprises a compounds of the formula 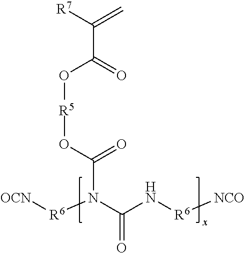
wherein
R⁵ is a divalent alkylene radical having 2 to 12 carbon atoms and optionally substituted with C₁ to C₄ alkyl groups and/or interrupted by one or more oxygen atoms,
R⁶ is a divalent alkylene or cycloalkylene radical having 2 to 20 carbon atoms and optionally substituted with C₁ to C₄ alkyl groups and/or interrupted by one or more oxygen atoms,
R⁷ is hydrogen or methyl, and
X is a positive number which on average is 2 up to 6.

10. The process according to claim 9, wherein R⁵ is selected from the group consisting of 1,2-ethylene, 1,2- or 1,3-propylene, 1,2-, 1,3- or 1,4-butylene, 1,1-dimethyl-1,2-ethylene, 1,2-dimethyl-1,2-ethylene, 1,5-pentylene, 1,6-hexylene, 1,8-octylene, 1,10-decylene, and 1,12-dodecylene.

11. The process according to claim 9, wherein R⁶ is selected from the group consisting of 1,6-hexylene,

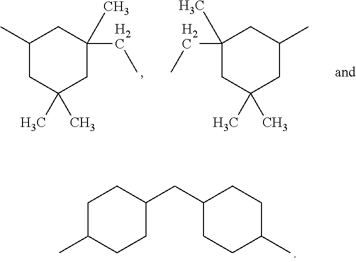

12. A radiation-curable coating material comprising at least one urethane (meth)acrylate according to claim 1 and, optionally, at least one radically polymerizable compound and, also optionally, at least one photoinitiator.

13. (canceled)

14. A method of coating a substrate comprising applying the radiation-curable coating material of claim 12 to a substrate selected from the group consisting of wood, paper, textile, leather, nonwoven, plastics surfaces, PVC, glass, ceramic, mineral building materials, molded cement blocks, fiber cement slabs, metals, or coated-metal substrates.
